(19) United States
(12) Patent Application Publication
(10) Pub. No.: US 2009/0105755 A1
(43) Pub. Date: Apr. 23, 2009

(54) APPARATUS AND METHOD FOR CONNECTING SPINAL FIXATION SYSTEMS TOGETHER

(75) Inventor: Marco D. CAPOTE, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedics, Inc., Warsaw, IN (US)

(21) Appl. No.: 11/876,021

(22) Filed: Oct. 22, 2007

(51) Int. Cl. A61B 17/70; A61B 17/00; A61B 17/56; A61B 17/04; A61B 17/58

(52) U.S. Cl. 606/246; 606/151; 606/64; 606/280; 606/286; 606/264; 606/301

(57) ABSTRACT

A connection system is provided that joins two or more spinal fixation plate systems together. In one form, a pair of bone bolts forms two docking points for a connecting member to attach to and brace two spinal fixation plate systems together. In another form, the connecting member is adjustable to allow for different dimensions between two spinal fixation plate systems. In another embodiment, a cap forms a dynamic connection between a bone bolt and a connecting member.

The present disclosure concerns orthopedic implants, such as those for the spine. Specifically, the present disclosure includes two or more spinal fixation systems attached to vertebrae, further these spinal fixation systems are linked together to treat and correct spinal deformities.

In the art of orthopedic surgery, a pair of elongated slotted plates or rods can be attached to bones to provide a more rigid support and alignment system. For example, in a procedure to fuse damaged vertebrae, the vertebrae are aligned as required by the surgeon. Each of the pair of plates are disposed along the vertebral column such that a first plate is offset a distance from a second plate. The plates are also fixed at selected points to various vertebrae along the length of the column by any number of fixation elements. In this way, such plates can provide support for or fixation of vertebrae or other tissues so that healing of trauma or surgical intervention, correction of deformation, or other therapy can occur.

### SUMMARY

Among other things, there are disclosed embodiments of orthopedic surgical apparatus including a pair of plate members, each plate member defining a respective slot, a first bone bolt connected to one of the plate members, a second bone bolt connected to the other of the plate members, and a connecting member connected to the bone bolts. The connecting member may have a first substantially circular end defining a first hole for accommodating at least a portion of the first bolt, and a second substantially circular end defining a second hole for accommodating at least a portion of the second bolt, with the ends having respective diameters. The connecting member further includes a linear central portion integral with the ends and having a longitudinal slot, with the central portion having a width that is less than the diameters of the ends. A pair of locking members are each adapted to hold the connecting member to one of the bolts and one of the plate members. The connecting member may have an extension portion and a receiving portion, with the extension portion being slidably accommodated within the receiving portion. Such an extension portion can include a pair of extending arms and such a receiving portion can include a pair of hollow receiver arms, each receiver arm sized to receive and to retain one of the extending arms. The connecting member may have upper and lower flat surfaces and curved side surfaces, and it may have a peg extending from one or both of said ends and substantially parallel to a longitudinal axis of its central portion. Such pegs may be rectangular in cross-section, and/or positioned along the longitudinal axis. A cap, which may be made of a silicone material, can be positioned between one of the plate members and the connecting member. The locking members may include an internally threaded nut, with the nuts having an upper internal hexagonal print and a substantially circular perimeter with a diameter that is slightly smaller than the diameter of the ends of the connecting member and larger than the width of said central portion of the connecting member. Dual-threaded members each having internal and external threads and a base flange may also be provided, with one of the dual-threaded members being threaded onto the first bolt over the first plate to at least partially anchor the first bolt to the first plate, and the other of the dual-threaded members being threaded onto the second bolt over the second plate to at least partially anchor the second bolt to the second plate. One of the locking members can be threaded onto the external threads of one of the dual-threaded members and the other of the locking members can be threaded onto the external threads of the other of the dual-threaded members.

Also disclosed are embodiments of orthopedic surgical apparatus including a connecting member for connecting separate orthopedic plate structures, with the connecting member having first and second substantially straight sides defining an axis and first and second curved ends, the first side being continuous with the first end and the second end, and the second side being continuous with the first end and the second end. The first side and second side define a slot, the first end defines a first hole and the second end defines a second hole, and the connecting member further including a first peg extending from the first end and a second peg extending from the second end, with the pegs having respective lengths suitable for attaching orthopedic implant structure to them. Such a connecting member can include an extension portion and a receiving portion, with the extension portion being slidably accommodated within the receiving portion. Such apparatus can also include a pair of plates, each plate having a first substantially straight side wall opposite a second substantially straight side wall and a pair of curved end walls separated from each other by the first and second side walls, and the side walls and end walls define a slot, as well as a first bone bolt connected to one of the plates through the slot of said first plate and a second bone bolt connected to the other of the plates through the slot of the second plate. The connecting member may be connected to the bolts, with its first hole positioned around the first bone bolt and its second hole positioned around the second bone bolt. Further, a pair of locking members may be provided, each adapted to hold the connecting member to one of the first bolt or the second bolt and one of the plates. Such locking member may include a nut having a set of external threads and a set of internal threads. The first bone bolt can include a threaded portion and the set of internal threads of the nut is adapted to thread with the threaded portion of the first bone bolt. The apparatus can also include a cap (which may be of flexible material) positioned between one of the plates and the connecting member. The connecting member can have upper and lower flat surfaces and curved side surfaces.

### DESCRIPTION OF THE DRAWINGS

FIG. 1 is a perspective view of an embodiment of a spinal fixation plate system.

FIG. 2 is a partially exploded perspective view of a spinal fixation plate system.

FIG. 3 is a perspective view of an assembled combination of the components of FIG. 2.

FIG. 4 is a partial cross-sectional view of the embodiment of FIG. 3, taken along lines 4-4 in FIG. 3 and viewed in the direction of the arrows

FIG. 5 is a top plan view of an embodiment of a connecting member.

FIG. 6 is a cross-sectional view of the embodiment of FIG. 5, taken along lines 6-6 in FIG. 5 and viewed in the direction of the arrows.

FIG. 7 is a perspective view of the embodiment of FIG. 5.

FIG. 8 is a top plan view of an embodiment of a connecting member.

FIG. 9 is a perspective view of the embodiment of FIG. 8.

FIG. 10 is a perspective view of an embodiment of a spinal fixation plate system.

FIG. 11 is a partially exploded perspective view of a spinal fixation plate system.

FIG. 12 is a partially exploded perspective view of a spinal fixation plate system.

FIG. 13 is a partially exploded perspective view of a spinal fixation plate system.

### DESCRIPTION OF THE ILLUSTRATED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the claims is thereby intended, such alterations and further modifications in the illustrated devices, and such further applications of the principles of the disclosure as illustrated therein, being contemplated as would normally occur to one skilled in the art to which the disclosure relates. One embodiment of the disclosure is shown in great detail, although it will be apparent to those skilled in the relevant art that some features that are not relevant to the present disclosure may not be shown for the sake of clarity.

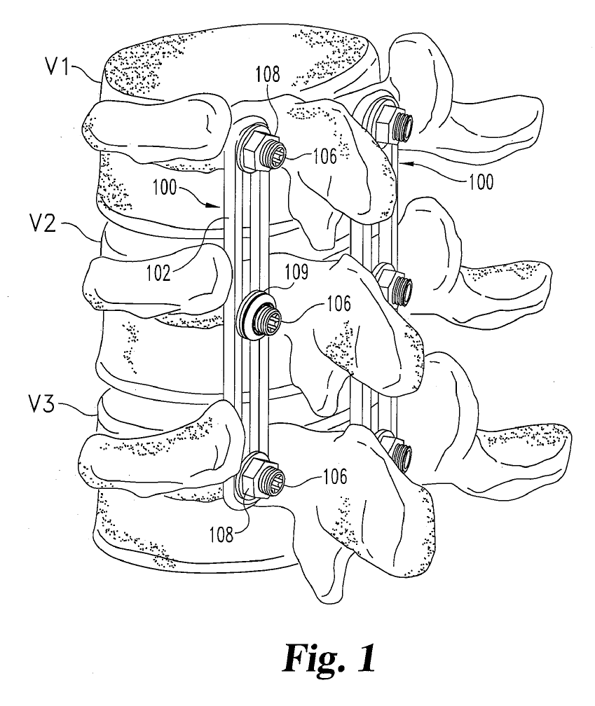

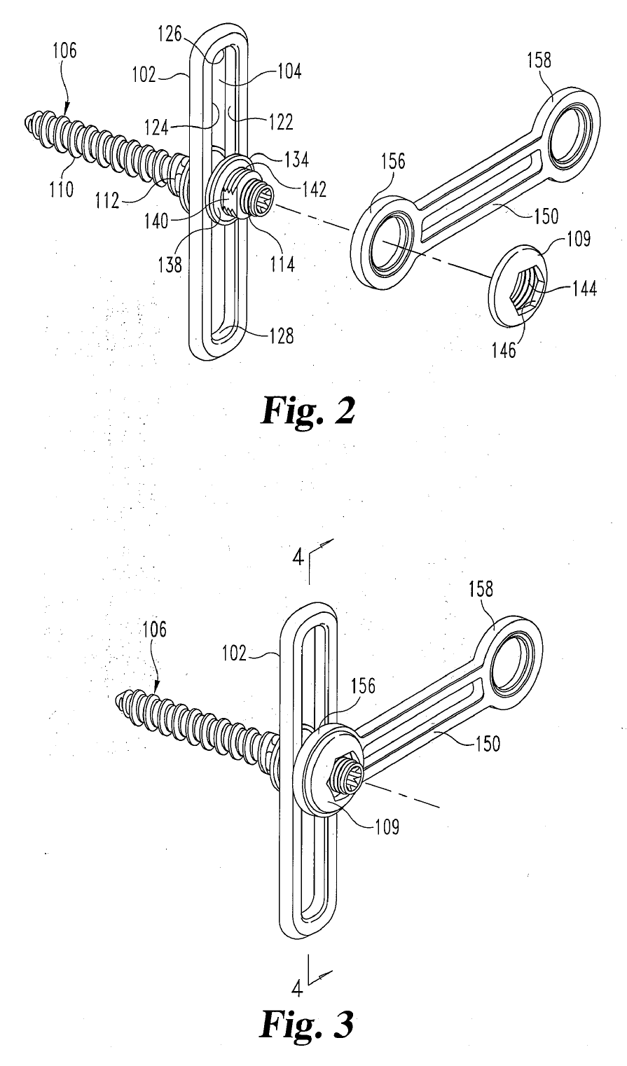

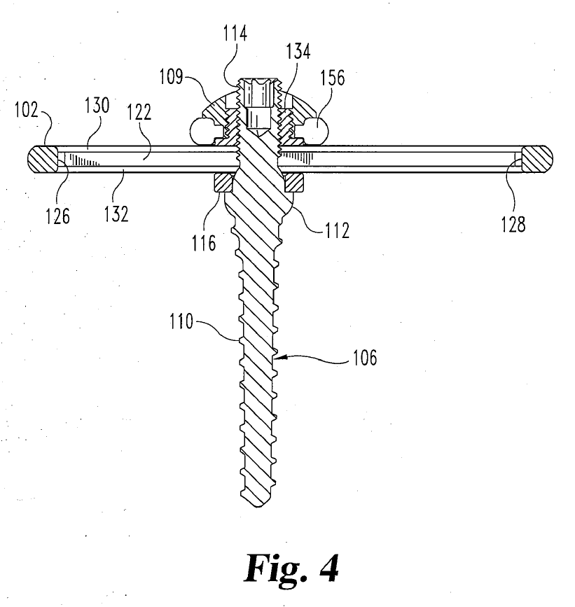

Referring generally to FIGS. 1, 2, 3, and 4, there is shown an embodiment of a spinal fixation plate system 100. Spinal fixation system 100 includes a plate 102 extending between vertebrae V1, V2 and V3 having an internal slot 104. A bolt 106 extends through slot 104 into the pedicle of vertebra V1 and is coupled to plate 102 by an internally threaded fastener 108. Also illustrated are two additional bolts 106, each bolt extending through slot 104 into the pedicles of vertebrae V2 and V3. In vertebra V2, bolt 106 is coupled to plate 102 by an internally threaded nut 109. In other embodiments, vertebrae V1, V2, and V3 can be attached to bolt 106 by a locking member, such as, fastener 108, nut 109, or other types of fixation elements. In the illustrated embodiment, there is a second spinal fixation plate system 100 attached to vertebrae V1, V2 and V3.

Bone bolt 106 includes a bone engaging threaded shaft 110, an enlarged seat area 112, and an externally threaded coupling shaft 114 having machine threads to receive a fixation element such as fastener 108 or nut 109. In the illustrated embodiment, disposed between seat 112 and plate 102, is an enlarged washer 116 adapted to engage the underside of plate 102. In the illustrated embodiment, bone bolt 106 has an external drive pattern on enlarged seat 112 and an internal hex drive pattern formed adjacent threads 114. Other embodiments of bolt 106 are contemplated as being within the scope of the present disclosure. For example, bolt 106 could include a bone-engaging hook rather than a threaded structure. In that embodiment, bone engaging threaded shaft 110 would be configured with a hook rather than an elongated threaded section.

Slot 104 in plate 102 is defined by opposing elongated side walls 122, 124 and opposing end walls 126, 128. The top of walls 122, 124, 126, and 128 forming slot 104 are chamfered or rounded to form surface 130, and the bottom of walls 122, 124, 126, and 128 are also chamfered to form surface 132. As shown, slot 104 has a substantially rectangular shape with rounded corners. Yet, in other embodiments, slot 104 can be shaped differently, such as, square, oval, circular, or any other shape the surgeon desires.

Spinal fixation system 100 further includes a coupling member 134 and cooperating internally threaded nut 109 to join plate 102 to a connecting member 150. Coupling member 134 has an enlarged flange 138 having a diameter larger than the width of slot 104 such that flange 138 engages plate 102. Coupling member 134 has an internally threaded bore configured to threadedly engage externally threaded shaft 114 of bone bolt 106 and an external drive surface 140 and an opposing drive surface (not shown). Drive surface 140 allows a tool to engage coupling member 134 and advance it along threaded shaft 114. The exterior of coupling member 134 has a series of external threads 142 interrupted by drive surface 140. Threaded nut 109 includes an internal drive pattern 144 and an internally threaded aperture 146. Internal drive pattern 144 is configured for threaded engagement with external threads 142.

Although not illustrated, it is contemplated that the spinal fixation systems described in this application can be configured for use with “Revision Fixation Plate and Method of Use”, application Ser. No. ______, filed Apr. 26, 2006, Attorney Docket No. 31132.461, which is hereby incorporated by reference.

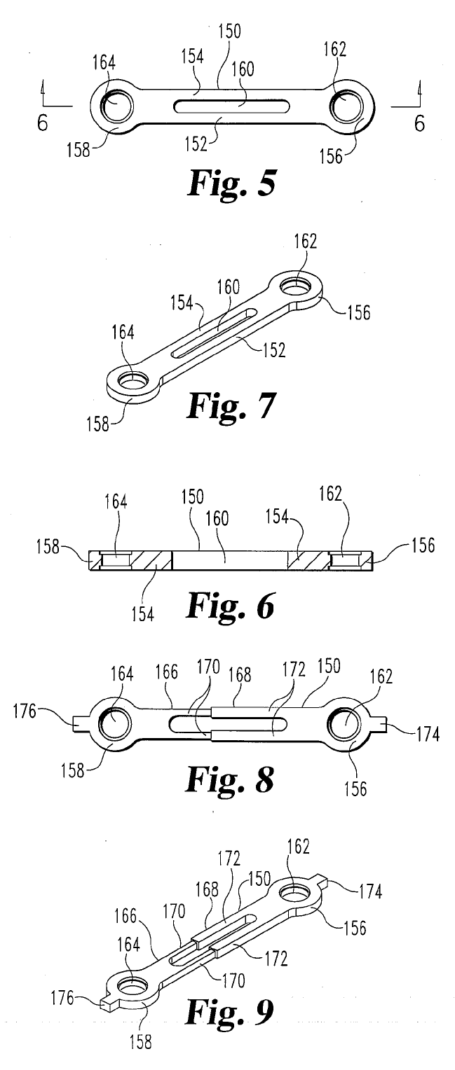

In FIGS. 5, 6, and 7, an embodiment of connecting member 150 is illustrated. In this embodiment, connecting member 150 is generally in the form of a “dogbone” shape having a first side 152, a second side 154, a first end 156, and a second end 158. In other embodiments, connecting member 150 may be oval, rectangular, or other shapes.

Each of sides 152 and 154 has a length between ends 156 and 158, and in the illustrated embodiment, sides 152 and 154 are substantially straight over their lengths between ends 156 and 158. In other embodiments, sides 152 and 154 may be curved or may be shaped independently of each other. In the illustrated embodiment, sides 152 and 154 have a rectangular cross-section perpendicular to a longitudinal axis of sides 152 and 154. In other forms, sides 152 and 154 can have circular, square, or oval cross-sections perpendicular to the longitudinal axis of sides 152 and 154. In the illustrated embodiment, sides 152 and 154 each have respective cross-sectional areas that are equal to each other. Sides 152 and 154 define or enclose a space or slot 160 in the illustrated embodiment. Space 160, in the illustrated embodiment, has an elongated oval shape; however, in other embodiments, space 160 can have a different shape, such as, rectangular, circular, or square, to name a few. In another embodiment, sides 152 and 154 form a continuous connecting member 150 without space 160.

In the illustrated embodiment, ends 156 and 158 have a substantially circular shape and ends 156 and 158 have a rectangular cross-section perpendicular to a central axis of each end. In other embodiments, ends 156 and 158 may be shaped differently or have a different cross-sectional shape, such as, circular, oval, square, or trapezoid, to name a few. Ends 156 and 158 define a first hole 162 and a second hole 164, respectively. In the illustrated embodiment, first hole 162 and second hole 164 are circular in shape; however, in other embodiments, first hole 162 and second hole 164 can be another shape adapted to accept bone bolts or other types of bone fixation devices. In the illustrated embodiment, first hole 162 and second hole 164 each have a stepped lower surface sized to matingly receive flange 138.

In a particular embodiment, illustrated in FIGS. 8 and 9, connecting member 150 includes an extension portion 166 and a receiving portion 168 to adjust the width of connecting member 150. Extension portion 166 and receiving portion 168 are shaped such that receiving portion 168 receives and holds extension portion 166. In the illustrated embodiment, extension portion 166 is configured to telescope into receiving portion 168, and receiving portion 168 includes a set screw to retain extension portion 166 at a desired location to fix the width of connecting member 150. In other embodiments, extension portion 166 may be retained in receiving portion 168 by other types of fixation systems. As illustrated, extension portion 166 includes a pair of extending arms 170, and receiving portion 168 includes a pair of hollow receiver arms 172. In this embodiment, extending arms 170 and receiver arms 172 are each rectangular in cross-sectional shape. In other embodiments, arms 170 and 172 can have a different cross-sectional shape, such as, circular, square, oval, or elliptical.

In the illustrated embodiment, in FIGS. 8 and 9, connecting member 150 includes a first peg 174 extending from first end 156 and a second peg 176 extending from second end 158. As shown, first peg 174 and second peg 176 are substantially parallel with elongated side walls 152 and 154. In this embodiment, first peg 174 and second peg 176 are similarly shaped and sized; however, in other embodiments, first peg 174 can be a different shape and/or size than second peg 176. As illustrated, first peg 174 and second peg 176 are each rectangular in cross-sectional shape; however, in other embodiments, first peg 174 and/or second peg 176 can be circular, triangular, or trapezoidal in cross-sectional shape. In other embodiments, connecting member 150 may include one peg extending from either first end 156 or second end 158. Yet in other embodiments, connecting member 150 may include multiple pegs extending from first end 156 and/or second end 158. First peg 174 and/or second peg 176 can be used for attachment of crosslinks, multi-axial screws, or other orthopedic devices to spinal fixation plate systems described in this application.

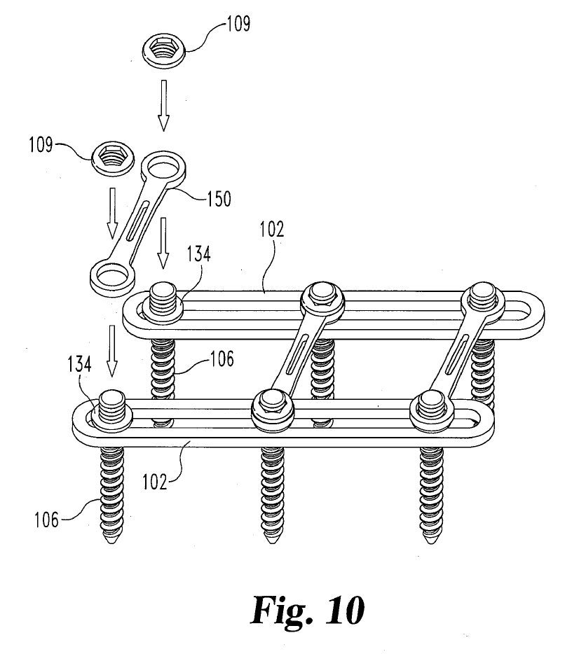

In the assembled configuration shown in FIGS. 3 and 10, connecting member 150 is positioned in substantial perpendicular alignment with plate 102. Coupling member 134 and projecting portion of threaded shaft 114 are received within either first hole 162 or second hole 164 of connecting member 150. Threaded nut 109 is threadedly advanced along external threads 142 such that nut 109 engages first end 156 or second end 158 of connecting member 150 and coupling member 134.

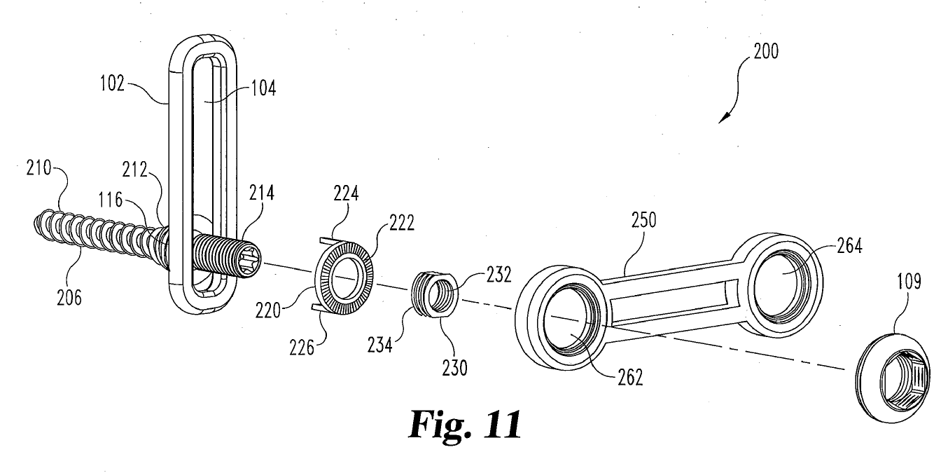

Referring to FIG. 11, there is shown another embodiment of a spinal fixation plate system 200. Plate 102 is attached to bone via a bone screw 206 having a bone engaging threaded shaft 210, an enlarged seat area 212, and an externally threaded coupling shaft 214 having machine threads to receive a fastener such as fastener 108 or nut 109. In the illustrated embodiment, disposed between seat 212 and plate 102 is enlarged washer 116 adapted to engage the underside of plate 102. A locking washer 220 includes a series of radially extending splines 222 projecting upwardly and a pair of downwardly projecting tabs 224 and 226. Locking washer 220 is positioned over threaded coupling shaft 214 with tabs 224 and 226 extending into slot 104 of plate 102. A dual threaded nut 230 is advanced along threaded coupling shaft 214 to engage a recessed area of washer 220 adjacent to splines 222 to thereby lock washer 220 to plate 102. Dual threaded nut 230 includes a series of internal threads 232 and a series of external threads 234. Internal threads 232 engage threaded coupling shaft 214 of bone screw 206. Connecting member 250 is similar to connecting member 150; however, connecting member 250 includes a series of radially extending splines corresponding to splines 222 formed on its bottom surface surrounding first hole 262 and second hole 264. Connecting member 250 is positioned over threaded post 214 such that splines on connecting member 250 are matingly interdigitated with splines 222. Internally threaded nut 109 is applied over threaded post 214 to lock the assembly in position. In the illustrated embodiment, plate 102 and connecting member 250 are positioned at substantially a right angle from each other; however, in other embodiments plate 102 may extend at another angle with respect to connecting member 250. While tabs 224 and 226 are shown in the illustrated embodiment to resist rotation about bone bolt 206, in other embodiments tabs 224 and 226 are removed and resistance to rotation of washer 220 against plate 102 is accomplished by other forms of compression onto plate 102, such as, a roughened surface on the washer bottom, frictional engagement, or an interference fit.

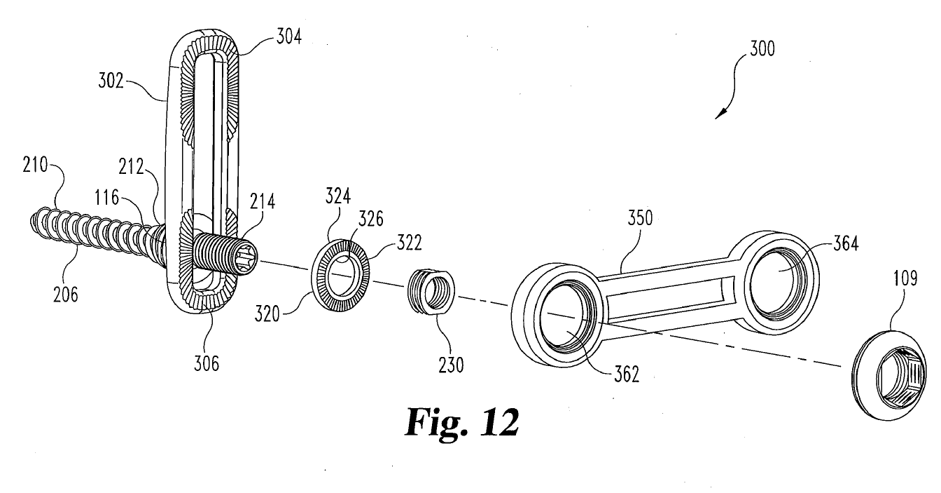

Illustrated in FIG. 12, there is shown another embodiment of a spinal fixation plate system 300. Plate 302 is similar to plate 102; however, plate 302 includes an upper surface 304 having a plurality of upwardly projecting splines 306. Plate 302 is attached to the bone via a bone screw 206. In the illustrated embodiment, disposed between seat 212 and plate 302 is enlarged washer 116 adapted to engage the underside of plate 302. A locking washer 320 includes a series of radially extending splines 322 projecting upwardly and a series of downwardly projecting splines 324. Locking washer 320 is advanced over threaded post 214 with downwardly projecting splines 324 configured to matingly engage splines 304. Dual threaded nut 230 is advanced along threaded post 214 to engage a recessed area 326 of washer 320 adjacent to splines 322 to thereby lock washer 320 to plate 302. Connecting member 350 is similar to connecting member 150; however, connecting member 350 has a series of radially extending splines corresponding to splines 322 formed on its bottom surface surrounding first hole 362 and second hole 364. Connecting member 350 is positioned over threaded nut 230 and post 214 such that the splines formed on the bottom of connecting member 350 are matingly interdigitated with splines 322. Internally threaded nut 109 is applied over threaded post 214 to lock the assembly in position. In the illustrated embodiment, plate 302 and connecting member 350 are positioned at substantially a right angle from each other; however, in other embodiments plate 302 may extend at a different angle with respect to connecting member 350. In other embodiments, radially projecting splines are replaced with a series of ridges extending transverse to the longitudinal axis of plate 302 or with a knurled surface and likewise the bottom of washer 320, or connecting member 350, is formed with a mating series of ridges or knurling.

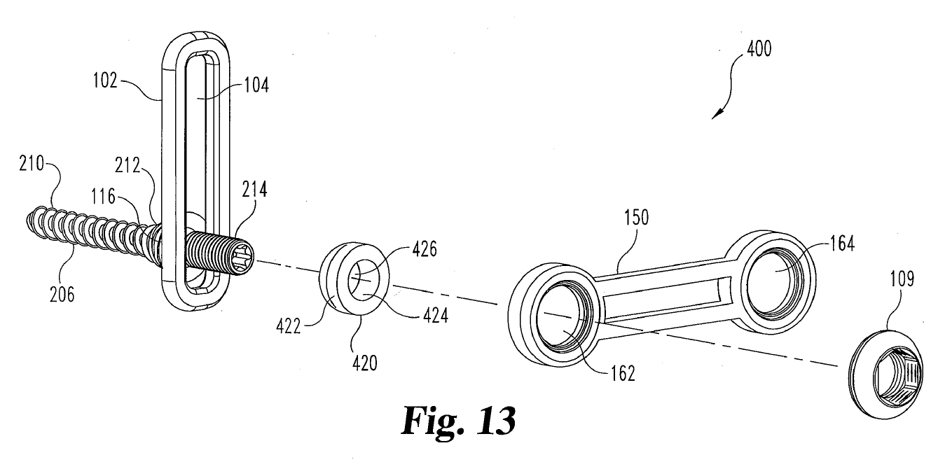

Referring to FIG. 13, there is shown another embodiment of a spinal fixation plate system 400. Plate 102 is attached to the bone via a bone screw 206. In the illustrated embodiment, disposed between seat 212 and plate 102 is enlarged washer 116 adapted to engage the underside of plate 102. A cap 420 having a wall 422 and defining a first opening 424 and a second opening 426 is positioned over threaded coupling shaft 214. Cap 420 is substantially parabolic as illustrated; however in other embodiments cap 420 may be shaped differently. Cap 420 is made of a flexible material. Some examples of flexible material include silicone or plastic. Cap 420 forms a dynamic connection between bone screw 206 and nut 109. Either first hole 162 or second hole 164 of connecting member 150 is positioned over threaded post 214 such that the lower surface of connecting member 150 contacts cap 420. Internally threaded nut 109 is applied over threaded post 214 to lock the assembly in position. In the illustrated embodiment, plate 102 and connecting member 150 are positioned at substantially a right angle from each other; however, in other embodiments plate 102 may extend at a different angle with respect to connecting member 150.

The connecting members as disclosed herein can accommodate different distances between the plates and orientations of the plates. The connecting members bridge the distance between the plates without interfering with the structures and nerves, e.g., the spinal cord and/or spinal processes associated with or adjacent to the spinal column. Additionally, the connecting members rigidly and securely connect the plates. Moreover, the connecting members can be adjustable. The fixation elements connect the connecting members to the pair of plates. Further, the fixation elements are configured to act as docking points for additional plates or rods that can be attached to the existing plates or rods in a future surgery, if necessary.

The devices of the present disclosure are constructed of any suitable biocompatible material; for example, in some embodiments, metals such as stainless steel and titanium, composites, ceramics, plastics, and polymers are used.

It is further contemplated that the present disclosure may be used with other types of spinal systems, such as, rod and screw systems, other plate and screw systems, and any spinal fixation or stabilization system to which the present disclosure may be connected. Additionally, the present disclosure may be used to connect any number of vertebrae, bone, or other tissue.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

What is claimed is:

1. An orthopedic surgical apparatus comprising:
a pair of plate members, each plate member defining a respective slot;
a first bone bolt connected to one of said plate members;
a second bone bolt connected to the other of said plate members;
a connecting member connected to said first bone bolt and said second bone bolt, said connecting member having a first substantially circular end defining a first hole for accommodating at least a portion of said first bone bolt, and a second substantially circular end defining a second hole for accommodating at least a portion of said second bone bolt, said ends having respective diameters, and said connecting member further including a linear central portion integral with said ends and having a longitudinal slot, said central portion having a width that is less than said diameters of said ends; and
a pair of locking members, each locking member adapted to hold said connecting member to one of said first bone bolt or said second bone bolt and one of said plate members.

2. The apparatus of claim 1, wherein said connecting member includes an extension portion and a receiving portion, said extension portion being slidably accommodated within said receiving portion.

3. The apparatus of claim 2, wherein said extension portion includes a pair of extending arms and said receiving portion includes a pair of hollow receiver arms, each receiver arm sized to receive and to retain one of said extending arms.

4. The apparatus of claim 1 wherein said connecting member has upper and lower flat surfaces and curved side surfaces.

5. The apparatus of claim 1, wherein said central portion of said connecting member has a longitudinal axis, and said connecting member includes a peg extending from one of said ends and substantially parallel to said axis.

6. The apparatus of claim 5, wherein said connecting member includes a peg extending from the other of said ends and substantially parallel to said axis.

7. The apparatus of claim 5, wherein said peg is rectangular in cross-section.

8. The apparatus of claim 5, wherein said peg is along said axis.

9. The apparatus of claim 1, further comprising a cap positioned between one of said plate members and said connecting member.

10. The apparatus of claim 9, wherein said cap is made of a silicone material.

11. The apparatus of claim 1, wherein each of said locking members comprises an internally threaded nut, said nuts having an upper internal hexagonal print and a substantially circular perimeter with a diameter, said nut diameter being slightly smaller than the diameter of the ends of the connecting member and larger than the width of said central portion of said connecting member.

12. The apparatus of claim 11, further comprising a pair of dual-threaded members each having internal and external threads and a base flange, one of said dual-threaded members being threaded onto said first bolt over said first plate to at least partially anchor said first bolt to said first plate, and the other of said dual-threaded members being threaded onto said second bolt over said second plate to at least partially anchor said second bolt to said second plate, wherein one of said locking members is threaded onto the external threads of one of said dual-threaded members and the other of said locking members is threaded onto the external threads of the other of said dual-threaded members.

13. An orthopedic surgical apparatus comprising:
a connecting member for connecting separate orthopedic plate structures, said connecting member having first and second substantially straight sides defining an axis and first and second curved ends, said first side being continuous with said first end and said second end, and said second side being continuous with said first end and said second end, said first side and second side defining a slot, said first end defining a first hole and said second end defining a second hole, and said connecting member further including a first peg extending from said first end and a second peg extending from said second end, said pegs having respective lengths suitable for attaching orthopedic implant structure to them.

14. The apparatus of claim 13, wherein said connecting member includes an extension portion and a receiving portion, said extension portion being slidably accommodated within said receiving portion.

15. The apparatus of claim 13, further comprising:
a pair of plates, each plate having a first substantially straight side wall opposite a second substantially straight side wall and a pair of curved end walls separated from each other by said first side and said second side walls, and said first side wall, said second side wall, and said pair of end walls defining a slot;
a first bone bolt connected to one of said plates through said slot of said first plate;
a second bone bolt connected to the other of said plates through said slot of said second plate;
said connecting member connected to said first bone bolt and said second bone bolt, said first hole positioned around said first bone bolt and said second hole positioned around said second bone bolt;
a pair of locking members, each locking member adapted to hold said connecting member to one of said first bone bolt or said second bone bolt and one of said plates.

16. The apparatus of claim 15, wherein said locking member includes a nut, said nut having a set of external threads and a set of internal threads.

17. The apparatus of claim 16, wherein said first bone bolt includes a threaded portion and said set of internal threads of said nut is adapted to thread with said threaded portion of said first bone bolt.

18. The apparatus of claim 15, further comprising a cap positioned between one of said plates and said connecting member.

19. The apparatus of claim 18, wherein said cap is made of a flexible material.

20. The apparatus of claim 13 wherein said connecting member has upper and lower flat surfaces and curved side surfaces.
